(19) United States
(12) Patent Application Publication
(10) Pub. No.: US 2003/0198625 A1
(43) Pub. Date: Oct. 23, 2003

(54) Electroporation-mediated transfection of the salivary gland

(75) Inventors: Hsien Tseng, San Francisco, CA (US); Michael J. Bennett, El Sobrante, CA (US); Stephan S. Rothman, Berkeley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA

(21) Appl. No.: 10/126,315

(22) Filed: Apr. 19, 2002

(51) Int. Cl. A61K 48/00; C12N 15/87

(52) U.S. Cl. 424/93.21; 435/461

(57) ABSTRACT

The present invention is directed toward a method of enhancing transfection efficiency by administering a nucleic acid to a salivary gland and electroporating the salivary gland.

### CROSS-REFERENCES TO RELATED APPLICATIONS

Not applicable.

### STATEMENT AS TO RIGHTS TO INVENTIONS MADE UNDER FEDERALLY SPONSORED RESEARCH AND DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK.

Not applicable.

### BACKGROUND OF THE INVENTION

The ability to replace defective or absent genes has attracted wide attention as a method to treat a variety of human diseases (Crystal, Science 270:404 (1995), Lever et al. Gene Therapy, Pearson Professional, New York p. 1-91 (1995), and Friedmann, Nature Med. 2:144 (1996)). Gene-based therapy can be a useful means to supply exogenous gene products to the circulatory system for the treatment of a wide range of systemic disorders that involve deficiencies in circulating proteins, such as hormones, growth factors, clotting proteins, and immunoglobulins (Lever et al. (1995) and Buckel, TiPS 17:450 (1996)), as well as a means of administering other polypeptide drugs. The success of this therapeutic application depends upon developing effective methods to deliver and express genes encoding proteins of interest in vivo. (Crystal (1995); Lever et al. (1995)).

There are currently two general methods for transferring exogenous genes into humans and other mammals: viral and non-viral. Both of these methods have their associated advantages and disadvantages involving either transfection efficiency or safety issues. For example, adenoviral vectors induce potent immune responses (Baum and O'Connell, Crit. Rev. Oral Biol. Med. 10(3):276 (1999)) and transfection efficiency is low when genes are transferred using non-viral delivery systems. Genetic manipulation of cells to express a protein for systemic delivery to the organism has been problematic. Thus, there is a need in the art to develop gene transfer techniques that enhance gene transfection efficiency in a safe manner.

### SUMMARY OF THE INVENTION

The present invention provides a method of efficient gene transfection via in vivo electroporation of salivary glands. One embodiment of the present invention provides a method for transfecting salivary gland cells by electroporation. A nucleic acid is administered to a salivary gland; and the salivary gland is electroporated. Administration may be by cannulation or injection. Administration may be via a salivary gland duct. The salivary gland may be a submandibular salivary gland, a parotid salivary gland, or a sublingual salivary gland. The nucleic acid may be operably linked to an expression control sequence. The nucleic acid may encode secreted alkaline phosphatase or luciferase. The nucleic acid may encode a therapeutic protein such as, for example, growth hormone, insulin, clotting factor VIII, clotting factor IX, erythropoietin, calcitonin, alpha-galactosidase, alpha-glucosidase, glucocerebrosidase, or immunoglobulin.

In another embodiment of the invention, the step of electroporating may comprise contacting and pulsing the salivary gland with an electrode comprising 2 needles. The step of electroporating may further comprise repositioning the electrode and contacting and pulsing the salivary gland with the electrode. The step of electroporating may comprise contacting the salivary gland with a first electrode in a first position and contacting the salivary gland with a second electrode in a second position and pulsing the salivary gland. If more than one electrode is used, the steps of contacting may be sequential or simultaneous.

The two needles may be about 1 cm apart or about 0.5 cm apart. The electrodes may emit an electric field strength from about 1 to about 1000 V/cm and a pulse length from about 1 to about 60 ms. The electrodes may emit an electric field strength from about 100 V/cm to about 200 V/cm and an electrical pulse length from about 10 ms to about 20 ms. The number of pulses may be from about 1 to about 30 pulses or from about five pulses to about six pulses.

In yet another embodiment of the invention, a formulant may be administered with the nucleic acid. The formulant may be divalent transition metal compounds, polyanionic compounds, or peptides. The divalent transition metal compound may be zinc halide, zinc oxide, zinc selenide, zinc telluride, zinc sulfate, zinc acetate, or zinc chloride. The polyanionic compound may be poly-L-glutamate. The formulant may be polyvinyl alcohol.

Other embodiments and advantages of the present invention will be apparent from the detailed description that follows.

### BRIEF DESCRIPTION OF THE DRAWINGS

FIG. 1 illustrates expression of luciferase in rat submandibular salivary glands. 48 hours after administration of the luciferase encoding DNA construct with or without electric pulse application, Luciferase activity was measured, and is expressed as Relative Light Units. Results are shown as the mean±SE. N for each group is in parentheses. ‘EP’ represents the application of electroporation treatment with an electric field strength of 100V/cm, a pulse length of 20 ms, and a total of 6 pulses. Each salivary gland received 2 sets of electroporation treatment with the same electric pulse parameters at the same time immediately after DNA delivery. The salivary glands of the control animals were not electroporated.

FIG. 2 illustrates levels of secreted alkaline phosphatase (SEAP) activity in rat submandibular salivary glands. 48 hours after introduction of the SEAP encoding construct (the injected plasmid DNA was mixed with 10 mM Zinc chloride), SEAP activity was measured. Bars represent the mean±SE, observed in the presence (EP) or the absence (control) of electric pulse stimulation; EP with or without PVA (0.32 M) in DNA containing solution. For electroporation, 200V/cm of electric field strength, 20 ms of pulse length with 6 pulses and 2 sets of electric stimulation were used. Expression level was 5294 pg/g and 2150 pg/g tissue for EP treated animals and 142 pg/g tissue for control animals (DNA alone). N for each group is in parentheses.

FIG. 3 illustrates expression of SEAP activity in the rat plasma from animals in which submandibular salivary glands were transfected. Concentrations of SEAP were 91 pg/ml and 26 pg/ml for the two EP groups and 2 pg/ml for the control group, indicating a 13-45.5-fold enhancement of gene expression by electroporation. Results are shown as the mean±SE. N for each group is in parentheses.

FIG. 4 illustrates expression of SEAP in the rat submandibular salivary glands. 48 hours after administration of the construct encoding SEAP (dissolved in 0.9% NaCl), SEAP activity was measured. Each of the three groups of animals was electroporated using different conditions, in which the spacing distance between 2 needles of the electrode and sets of electrical stimulation varied; but with the same electric field strength (200V/cm) and pulse length (20 ms) for 6 pulses. SEAP activities in submandibular gland between groups (EP-1, EP-2, and EP-3) were 24, 42, and 203 pg per g tissue respectively. Data are shown as the mean±SE. N for each group is in parentheses.

### DETAILED DESCRIPTION OF THE INVENTION

I. Introduction

The present invention is based on the surprising discovery that electroporation enhances DNA uptake into salivary gland cells thus increasing the efficiency of gene transfer. In particular, electroporation-mediated in vivo gene transfer systems have provided a new non-viral tool for gene transfer.

Recently, salivary glands have been the target of gene transfer experiments aimed at developing clinical applications to the treat salivary disorders or diseases involving systemic protein deficiencies (U.S. Pat. No. 6,255,289, U.S. Pat. No. 6,004,944, U.S. Pat. No. 5,885,971, U.S. Pat. No. 5,827,693, Baccaglini et al., J. Gene. Med. 3:82 (2001), Baum et al., Int. J. Oral Maxillofac. Surg. 29:163 (2000), Baum and O'Connell, Crit. Rev. Oral Biol. Med. 10(3):276 (1999), He et al., Gene Therapy 5:537 (1998), and Mastrangeli et al., Am. J. Physiol. 266:G1146 (1994)). Salivary glands are a good target for in vivo gene transfer because of their exocrine gland characteristics. The main secretory duct can conveniently be used to easily access the major salivary glands. The majority of salivary parenchymal cells can be transfected this way, thus these glands are capable of producing and secreting therapeutic proteins by both exocrine and endocrine secretory pathways. Moreover, expression of therapeutic proteins can be regulated physiologically in response to meal, chewing or different neural and hormonal stimulation. (See, e.g., U.S. Pat. No. 5,837,693). Expressed therapeutic proteins may be secreted into the saliva or into the bloodstream. (See, id.).

II. Definitions

As used herein, the following terms have the meanings ascribed to them below unless otherwise specified.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleotides may be referred to by their commonly accepted single-letter codes. These are A, adenine; C, cytosine; G, guanine; and T, thymine (DNA), or U, uracil (RNA).

The term “codon” refers to a sequence of nucleotide bases that specifies an amino acid or represents a signal to initiate or stop a function. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605 (1985); Rossolini et al., Mol. Cell. Probes 8:91 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified through post translational modification, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

Each of the following eight groups contains amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or “detectable label” is a composition detectable by spectroscopic, photochemical, biochemical, immunochemical, or chemical means. For example, useful labels include radioisotopes (e.g., ³H, ³⁵S, ³²p, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “salivary gland” is a gland of the oral cavity which secretes saliva, including the glandulae salivariae majores of the oral cavity (the parotid, sublingual, and submandibular glands) and the glandulae salivariae minores of the tongue, lips, cheeks, and palate (labial, buccal, molar, palatine, lingual, and anterior lingual glands).

A nucleic acid may be administered to the salivary gland with or without a “formulant,” i.e., a substance that enhances transfection efficiency. Suitable formulants include, for example, divalent transition metals and polyanionic compounds. “Divalent transitions metal compounds” refer to compounds comprising a divalent transition metal, such as, for example, zinc, copper, cobalt, or nickel. “Polyanionic compounds refer to compounds comprising one or more anionic units.

A nucleic acid administered to the salivary gland may be encapsulated in a liposome (or other cationic, anionic, or neutral polymer) formulation.

A “therapeutic protein” or “therapeutic nucleic acid” is any protein or nucleic acid that provides a therapeutic or prophylactic effect. A therapeutic protein may be naturally occurring or produced by recombinant means. A “therapeutically effective amount” of a nucleic acid or protein is an amount of nucleic acid or protein sufficient to provide a therapeutic or prophylactic effect in a subject. Such therapeutic or prophylactic effects may be local or systemic. Therapeutic and prophylactic effects include, for example, eliciting or modulating an immune response. Selby et al. (2000) J. Biotechnol. 83(1-2):147-52. Immune responses include humoral immune responses and cell-mediated immune responses.

“Electroporation” involves contacting cells, tissues, glands, or organs with electrodes and “pulsing” the cells, tissues, glands, or organs, i.e., passing an electric signal through the tissues, glands, or organs via the electrode. One preferred embodiment of the present invention comprises contacting a salivary gland with an electrode and “pulsing” the salivary gland. After contacting and pulsing the salivary gland, electrodes may be “repositioned” to come into contact with the same or different position on the salivary gland. After repositioning of the electrode, the salivary gland may be pulsed again. “Electrodes” that can be used to contact the cells, tissues, glands, or organs, include needles, laparoscopic needles, probes, needles with paddles, and needles with flat plates or calipers. Electrodes may comprise individual needles, laparoscopic needles, probes, needles with paddles, and flat plates or may comprise an array of multiple needles, e.g. 2, 4, 6, 8, 10, 12, 14, 16, 18, 20, 22, 24, 26, 28, or 30 needles, laparoscopic needles, probes, needles with paddles, and needles with flat plates or calipers. “Contacting” includes, placing the electrodes at or near the cells, tissues, glands, or organs; touching the cells, tissues, glands, or organs with the electrodes, or penetrating the tissues, glands or organs with the electrodes.

III. Nucleic Acids

According to the methods of the present invention, nucleic acids can be administered to a subject prior to, simultaneous with, or subsequent to electroporation. Nucleic acids that may be administered include, for example, nucleic acids encoding proteins, therapeutic proteins, antibodies, peptides, cyclic peptides, nucleic acids, RNAi, antisense nucleic acids, and ribozymes.

In a typical embodiment, nucleic acids that can be used in the present invention are those encoding therapeutic proteins that may be useful for treating or preventing a disease or disorder in a subject. Nucleic acids administered according to the methods of the present invention may encode proteins that have local or systemic effects. Proteins encoded by nucleic acids administered according to the methods of the present invention may be used, for example, to treat or prevent any disorder amenable to treatment or prevention by expression of a therapeutic protein into the blood stream, by secretion of a therapeutic protein to the gastrointestinal tract (e.g. by secretion of the protein into the saliva), or by expression of the therapeutic protein by the transfected cell, tissue, gland, or organ. The subject may be a mammal such as, for example, a mouse, a rat, a guinea pig, a cat, a dog, a sheep, a goat, a cow, a horse, a non-human primate, or a human; or a non-mammal, such as, for example, a frog, a toad, a lizard, a snake, a turtle, a tortoise, or a salamander.

A. Diseases and Disorders

The disease or disorder to be prevented or treated include autoimmune disorders, blood disorders, cardiovascular disorders, central nervous system disorders, gastrointestinal disorders, metabolic disorders, neoplastic diseases, pulmonary disorders, and bacterial and viral diseases.

Autoimmune disorders that can be treated according to the methods of the present invention, include, for example, arthritis, diabetes, systemic lupus erythematosus, or Grave's disease. Blood disorders that can be treated according to the methods of the present invention, include, for example, anemia sickle cell anemia, a globin disorder, or a clotting disorder such as hemophilia. Cardiovascular disorders that can be treated or prevented according to the methods of the present invention include, for example, high blood pressure, high cholesterol, and angina. Central nervous system disorders that can be treated according to the methods of the present invention, include, for example, Parkinson's disease, Alzheimer's disease, multiple sclerosis, and Lou Gehrig's disease. Gastrointestinal disorders that can be treated according to the methods of the present invention include esophageal reflux, lactose deficiency, and defective vitamin B12 absorption. Metabolic disorders that can be treated according to the methods of the present invention, include, for example, enzyme deficiencies, obesity, lysosomal storage disease, Hurler's disease, Scheie's disease, Hunter's disease, Sanfilippo diseases, Morqio diseases, Maroteaux-Lamy disease, Sly disease, or dwarfism. Neoplastic diseases that can be treated or prevented according to the methods of the present invention, include, for example, colon cancer, stomach cancer, liver cancer, pancreatic cancer, lung cancer, breast cancer, skin cancer, leukemia, lymphoma, or myeloma. Pulmonary disorders that can be treated according to the methods of the present invention include, for example, cystic fibrosis, emphysema, or asthma. Bacterial diseases that can be treated or prevented according to the methods of the present invention, include, for example diphtheria, Lyme disease, meningitis, food poisoning, or pneumonia. Viral diseases that can be treated or prevented according to the methods of the present invention, include, for example, HIV, Epstein Barr virus, herpes simplex virus, hepatitis A, hepatitis B, hepatitis, C, and hepatitis E, mumps, measles, polio, or chicken pox.

B. Therapeutic Proteins

Suitable therapeutic proteins encoded by nucleic acids administered according to the methods of the present invention include, for example, growth hormones, clotting factors such as, lysosomal enzymes, plasma proteins, plasma protease inhibitors, proteases, protease inhibitors, hormones, pituitary hormones, growth factors, somatomedins, gonadotrophins, apolipoproteins, insulinotrophic hormones, immunoglobulins, chemotactins, chemokines, interleukins, interferons, cytokines, fusion proteins, and antigens, such as, for example, viral antigens, bacterial antigens, fungal antigens, parasitic antigens, or antigens overexpressed on neoplastic cells.

Exemplary proteins suitable for use according to the methods of the present invention include, for example, insulin, insulintropin, glucagon, glucagon-like peptide (GLP), human growth hormone (hGH), bovine growth hormone (bGH), factor VIII and factor IX, erythropoietin (EPO), antithrombin III, thrombopoietin (TPO), calcitonin, α-galactosidase, α-glucosidase, glucocerebrosidase, β-glucuronidase, parathyroid like hormone (PTH), fibroblast growth factor (FGF), insulin-like growth factor (IGF), neurite growth factor (NGF), epidermal growth factor (EGF), transforming growth factor (TGF), granulocyte colony stimulating factor (G-CSF), granulocyte macrophage colony stimulating factor (GM-CSF), a interferon, γ-interferon, IL-1, IL-1 RA, IL-2, IL-4, IL-5, IL-10, IL-12, phenylalanine ammonia lyase, arginase, L-asparaginase, uricase, platelet derived growth factor (PDGF), brain derived neurite factor (BDNF), purine nucleotide phosphorylase, tumor necrosis factor (TNF), lipid-binding proteins (lbp), α-1-antitrypsin, apolipoprotein B-48, apolipoprotein Al₂, tissue plasminogen activator (tPA), urokinase, streptokinase, superoxide dismutase (SOD), catalase, adenosine deamidase, cholecystokinin (cck), ob gene product, vasoactive intestinal peptide (VIP), gastric inhibitory peptide (GIP), somatostatin, pepsin, trypsin, chymotrypsin, elastase, carboxypeptidase, lactase, sucrase, and intrinsic factor.

C. Cloning Methods for the Isolation of Nucleotide Sequences Encoding Nucleic Acids

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

In general, the nucleic acid sequences encoding therapeutic proteins and related nucleic acid sequence homologues are cloned from cDNA and genomic DNA libraries or isolated using amplification techniques with oligonucleotide primers. For example, nucleic acid sequences encoding therapeutic proteins are typically isolated from nucleic acid (genomic or cDNA) libraries by hybridizing with a nucleic acid probe, the sequence of which can be derived from the nucleic acid sequence of protein of interest.

Nucleic acids encoding therapeutic proteins can also be isolated from expression libraries using antibodies as probes. Polymorphic variants, alleles, and interspecies homologues that are substantially identical to the therapeutic protein can be isolated using nucleic acid probes and oligonucleotides under stringent hybridization conditions, by screening libraries. Alternatively, expression libraries can be used to clone polymorphic variants, alleles, and interspecies homologues, by detecting expressed homologues immunologically with antisera or purified antibodies made against the therapeutic protein, which also recognize and selectively bind to the homologue of the therapeutic protein.

To make a cDNA library, mRNA from a therapeutic protein may be purified from an appropriate source. The mRNA is then made into cDNA using reverse transcriptase, ligated into a recombinant vector, and transfected into a recombinant host for propagation, screening and cloning. Methods for making and screening cDNA libraries are well known (see, e.g., Gubler & Hoffman, Gene 25:263 (1983); Sambrook et al., supra; Ausubel et al., supra).

For a genomic library, the DNA is extracted from the tissue and either mechanically sheared or enzymatically digested to yield fragments of about 12-20 kb. The fragments of interest are then separated by gradient centrifugation from fragments of undesired sizes and are constructed in bacteriophage lambda vectors. These vectors and phage are packaged in vitro. Recombinant phage are analyzed by plaque hybridization as described in Benton & Davis, Science 196:180 (1977). Colony hybridization is carried out as generally described in Grunstein et al., Proc. Natl. Acad. Sci. USA., 72:3961 (1975).

An alternative method of isolating nucleic acids encoding therapeutic proteins and their homologues combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of the therapeutic protein directly from mRNA, from cDNA, from genomic libraries or cDNA libraries. Restriction endonuclease sites can be incorporated into the primers. Genes amplified by the PCR reaction can be purified from agarose gels and cloned into an appropriate vector.

Amplification techniques using primers can also be used to amplify and isolate DNA or RNA encoding a protein of interest. These primers can be used, e.g., to amplify either the full length sequence or a probe of one to several hundred nucleotides, which is then used to screen a cDNA library for full-length protein.

Synthetic oligonucleotides can be used to construct recombinant genes encoding therapeutic proteins for expression of the protein. This method is performed using a series of overlapping oligonucleotides usually 40-120 bp in length, representing both the sense and non-sense strands of the gene. These DNA fragments are then annealed, ligated and cloned. Alternatively, amplification techniques can be used with precise primers to amplify a specific subsequence of the gene encoding a therapeutic protein. The specific subsequence is then ligated into an expression vector.

Nucleic acids encoding therapeutic proteins are typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding therapeutic proteins comprise a nucleic acid sequence encoding a therapeutic protein and subsequences, interspecies homologues, alleles and polymorphic variants thereof.

D. Expression of Nucleic Acids

To obtain high level expression of a cloned gene, such as those cDNAs encoding a suitable therapeutic protein, one typically subclones the gene encoding the protein into an expression vector that contains a strong promoter to direct transcription, a transcription/translation terminator, and if for a nucleic acid encoding a protein, a ribosome binding site for translational initiation. Suitable promoters are well known in the art and described, e.g., in Sambrook et al. and Ausubel et al. Eukaryotic expression systems for mammalian cells are well known in the art and are also commercially available. Kits for such expression systems are commercially available.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

The nucleic acid comprises a promoter to facilitate expression of the nucleic acid within a salivary gland cell, more preferably a parotid gland cell, even more preferably a submandibular salivary gland cell. Suitable promoters include strong, eukaryotic promoter such as, for example promoters from cytomegalovirus (CMV), mouse mammary tumor virus (MMTV), Rous sarcoma virus (RSV), and adenovirus. More specifically, suitable promoters include the promoter from the immediate early gene of human CMV (Boshart et al., Cell 41:521 (1985)) and the promoter from the long terminal repeat (LTR) of RSV (Gorman et al., Proc. Natl. Acad. Sci. USA 79:6777 (1982)).

Salivary gland specific promoters may also be used in accordance with the present invention and include, for example, salivary a-amylase promoters and mumps viral gene promoters which are specifically expressed in salivary gland cells. Multiple salivary α-amylase genes, have been identified and characterized in both mice and humans (see, for example, Jones et al., Nucleic Acids Res., 17(16):6613 (1989); Pittet et al., J. Mol. Biol. 182:359 (1985); Hagenbuchle et al., J. Mol. Biol., 185:285 (1985); Schibler et al., Oxf. Surv. Eukaryot. Genes 3:210 (1986); and Sierra et al., Mol. Cell. Biol., 6:4067-(1986) for murine salivary α-amylase genes and promoters; Samuelson et al., Nucleic Acids Res., 16:8261 (1988); Groot et al., Genomics, 5:29 (1989); and Tomitaetal., Gene, 76:11 (1989) for human salivary α-amylase genes and their promoters). The promoters of these α-amylase genes direct salivary gland specific expression of their corresponding α-amylase encoding DNAs. These promoters may thus be used in the constructs of the invention to achieve salivary gland-specific expression of a nucleic acid of interest. Sequences which enhance salivary gland specific expression are also well known in the art (see, for example, Robins et al., Genetica 86:191 (1992)).

For eukaryotic expression (e.g., in a salivary gland cell), the construct may comprise at a minimum a eukaryotic promoter operably linked to a nucleic acid operably linked to a polyadenylation sequence. The polyadenylation signal sequence may be selected from any of a variety of polyadenylation signal sequences known in the art, such as, for example, the SV40 early polyadenylation signal sequence. The construct may also include one or more introns, which can increase levels of expression of the nucleic acid of interest, particularly where the nucleic acid of interest is a cDNA (e.g., contains no introns of the naturally-occurring sequence). Any of a variety of introns known in the art may be used.

Other components of the construct may include, for example, a marker (e.g., an antibiotic resistance gene (such as an ampicillin resistance gene)) to aid in selection of cells containing and/or expressing the construct, an origin of replication for stable replication of the construct in a bacterial cell (preferably, a high copy number origin of replication), a nuclear localization signal, or other elements which facilitate production of the nucleic acid construct, the protein encoded thereby, or both.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

Some expression systems have markers that provide gene amplification such as thymidine kinase, hygromycin B phosphotransferase, and dihydrofolate reductase.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

E. Liposomal Formulations

The nucleic acids may be in a liposomal preparation when they are administered to the salivary gland according to the methods of the present invention. Liposomal preparations suitable for use in the present invention include, for example, cationic, anionic, and neutral preparations. Liposomes suitable for use in the present invention include, for example, multilamellar vesicles (MLVs), small unilamellar vesicles (SUVs), or large unilamellar vesicles (LUVs). Commonly used methods for making liposomes include Ca²⁺-EDTA chelation (Papahadjopoulos, et al., Biochim. Biophys. Acta 394:483 (1975); Wilson, et al., Cell 17:77 (1979)); ether injection (Deamer and Bangham, Biochim. Biophys. Acta 443:629 (1976); Ostro, et al., Biochem. Biophys. Res. Commun. 76:836 (1977); Fraley, et al., Proc. Natl. Acad. Sci. USA 76:3348 (1979)); detergent dialysis (Enoch and Strittmatter, Proc. Natl. Acad. Sci. USA 76:145 (1979)) and reverse-phase evaporation (REV) (Fraley, et al., J. Biol. Chem. 255:10431 (1980); Szoka and Papahadjopoulos, Proc. Natl. Acad. Sci. USA 75:145 (1978); Schaefer-Ridder, et al., Science 215:166 (1982)).

Suitable cationic lipids, include, for example, DOTAP, DOTMA, DDAB, L-PE, and the like. Liposomes containing a cationic lipid, such as {N(1-2-3-dioleyloxy) propyl}-N,N,Ntriethylammonium} (DOTMA), dimethyl dioctadecyl ammonium bromide (DDAB), or 1, 2dioleoyloxy-3-(trimethylammonio) propane (DOTAP) or lysinylphosphatidylethanolamine (L-PE) and a second lipid, such as distearoylphosphatidylethanolamine (DOPE) or cholesterol (Chol). DOTMA synthesis is described in Felgner, et al., Proc. Nat. Acad. Sciences USA 84:7413 (1987). DOTAP synthesis is described in Stamatatos, et al., Biochemistry 27:3917 (1988). DOTMA:DOPE liposomes is commercially available from, for example, BRL. DOTAP:DOPE liposomes is commercially available from Boehringer Mannheim. Cholesterol and DDAB are commercially available from Sigma Corporation. DOPE is commercially available from Avanti Polar Lipids. DDAB:DOPE is commercially available from Promega.

Additionally, complexing the cationic lipid with a second lipid, primarily either cholesterol or DOPE can maximize transgene expression in vivo. For example, mixing cholesterol instead of DOPE with DOTAP, DOTMA, or DDAB may substantially increase transgene expression in vivo.

Anionic and neutral liposomes are commercially available, such as from Avanti Polar Lipids (Birmingham, Ala.), or can be easily prepared using commercially available materials, such as, for example, phosphatidylcholine, cholesterol, phosphatidylethanolamine, dioleoylphosphatidylcholine (DOPC), dioleoylphoshatidylethanolamine (DOPE). Methods for making liposomes using these materials are well known in the art.

F. Administration of Nucleic Acids

According to the present invention, the nucleic acid may be administered according to any means known in the art. Suitable methods of administration of the nucleic acid to the cells, tissues, glands, or organs include, for example, cannulation or injection of the nucleic acid into the cells, tissues, glands, or organs using a syringe, cannula, catheter, or shunt. In a preferred embodiment, the nucleic acid is administered to a salivary gland. In a particularly preferred embodiment, the nucleic acid is administered to a salivary gland through a salivary gland duct for retroductal delivery. The type of syringe used is not a critical part of the invention. One of skill in the art will appreciate that multiple types of syringes may be used to administer nucleic acids according to the present invention. Suitable types of syringes include, for example, an aspirating syringe, a removable needle syringe, a modified microliter syringe, a microliter syringe, a gastight syringe, a sample lock syringe, a threaded plunger syringe.

Delivery of the nucleic acid may be via gravity or an assisted delivery system. Suitable assisted delivery systems include controlled release pumps, time release pumps, osmotic pumps, and infusion pumps. The particular delivery system or device is not a critical aspect of the invention. One of skill in the art will appreciate that multiple types of assisted delivery systems may be used to delivery nucleic acids according to the methods of the present invention. Suitable delivery systems and devices are described in U.S. Pat. Nos. 5,492,534, 5,562,654, 5,637,095, 5,672,167, and 5,755,691. One of skill in the art will also appreciate that the infusion rate for delivery of the nucleic acid may be varied. Suitable infusion rates may be from about 0.005 ml/min to about 1 ma/minute, preferably from about 0.01 ml/min to about 0.8 mmin., more preferably from about 0.025 ml/min. to about 0.6 Ml/min. It is particularly preferred that the infusion rate is about 0.05 ml/min.

In accordance with the present invention, the nucleic acid may be administered alone or with a formulant that enhances transfection efficiency. Suitable formulants include, for example, divalent transition metals, polyanionic compounds, and peptides. Suitable divalent transition metal compounds include, for example, zinc halide, zinc oxide, zinc acetate, zinc selenide, zinc telluride, and zinc sulfate. Preferred suitable divalent transition metal compounds include, for example, ZnCl₂, CuCl₂, CoCl₂, NiCl₂, and MgSO₄ (Shiokawa et al., Biochem J. 326:675 (1997) and Torriglia et al., Biochimie 79:435 (1997)). Other suitable divalent transition metals are described in U.S. patent application Ser. No. 09/487,089, filed Jan. 19, 2000, U.S. patent application Ser. No. 09/766,320, filed Jan. 18, 2001, and International Publication WO 01/52903, filed Jan. 19, 2001. Suitable polyanionic compounds include, for example, poly-L-glutamate. Suitable peptides include, for example, ID2 and peptides based on it such as, for example ID2-2, ID2-3, ID2-4 (Sperinde et al., J. Gene Med. 3:101 (2001)). Other suitable formulants include, for example, polyvinyl alcohol and nuclease inhibitors (Glasspool-Malone, et al. Mol. Ther. 2(2): 140 (2000)), sodium citrate, and G-actin (Shiokawa et al. (1997), supra).

IV. Electroporation

According to the present invention, electroporation is used to enhance the efficiency of gene transfer after administration of a nucleic acid to a cell, tissue, gland or organ. In a particularly preferred embodiment, the nucleic acid is administered to a salivary gland before electroporation. The use of electroporation in gene delivery is described in Somiari and Glasspool-Malone, Mol. Ther. 2(3):178 (2000). Electroporation involves contacting cells, tissues, glands, or organs with an electrode comprising at least two needles and pulsing an electric signal through the cells, tissues, glands, or organs via the electrode. In a particularly preferred embodiment, a salivary gland is contacted with the electrode. The cells, tissues, glands, or organs may be contacted with more than two electrodes according to the methods of the present invention. If the cells, tissues, glands, or organs are contacted with more than one electrode, the contact may be simultaneous or sequential. The cells, tissues, glands, or organs may be contacted with the electrodes in multiple positions in accordance with the methods of the present invention. For example, the electrodes may be positioned vertically, longitudinally, or horizontally to come in contact with the salivary gland. The electrodes may also be positioned at angles to each other to come into contact with the cells, tissues, glands, or organs. Suitable angles include, for example, 45 degrees, 60 degrees, 75 degrees, 90 degrees, 120 degrees, 160 degrees, or 180 degrees. Preferably, the electrodes are positioned to ensure that the entire salivary gland is pulsed. One of skill in the art would understand that the position of the electrodes may be adjusted as needed to create an electric field that will extend throughout the entire salivary gland upon pulsing. The cells, tissues, glands, or organs may be contacted with electrodes that include, for example, needles, laparoscopic needles, probes, needles with paddles, needles with rotating paddles, and needles with flat plates or calipers. Methods of electroporation are described in U.S. Pat. Nos. 6,233,482, 6,135,990, 5,993,434, and 5,704,908). Electrodes may comprise individual needles, laparoscopic needles, probes, needles with paddles, and flat plates or may comprise an array of multiple needles, laparoscopic needles, probes, needles with paddles, and flat plates. One of skill in the art will appreciate that the space between 2 needles on the same electrode may be varied. The space between two needles may be, for example, about 0.1, 0.25, 0.4, 0.5, 0.6, 0.75, 1, 1.5, 2, 2.5, 3, 3.5, 4, 4.5, or 5 cm. Electrodes and electrode arrays are described in WO 98/47562. Other configurations of the electrodes and electrode arrays for example, angle or shape of needle array, may be used to meet particular size and access needs according to the present invention.

Factors to consider in determining suitable electroporation conditions include: electric field strength, pulse duration, pulse number, and pulse frequency. One of skill in the art will understand that appropriate values for each of these factors, i.e., values that enhance transfection efficiency, can be determined by standard means known in the art, i.e., without undue experimentation. (See, e.g., Canatella and Prausnitz, Gene Therapy 8:1464 (2001)). The electrode may emit an electric field strength from about 1 to about 1000 V/cm, from about 25 to about 750 V/cm, from about 50 to about 500 V/cm, form about 60 to about 300 V/CM or from about 75 to about 250 V/cm. The pulse length may be from about 1 to about 60 ms, from about 2 to about 50 ms, from about 4 to about 40 ms, from about 5 to about 30 ms, or from about 7 to about 25 ms. For example, a suitable electric field strength is typically from about 100 V/cm to about 200 V/cm and a suitable electrical pulse length is typically from about 10 ms to about 20 ms. A suitable number of pulses is typically from about 1 to about 30 pulses, from about 2 to about 20 pulses, from about 4 to about 15 pulses, from about 5 to about 12 pulses, preferably from about 5 pulses to about 6 pulses.

Suitable signal generators for electroporation are commercially available and include, for example, an Electro Cell Manipulator Model ECM 600 (Genetronics, Inc., San Diego, Calif.), an Electro Cell Manipulator Model ECM 830 (BTX, San Diego, Calif.), an ElectroSquarePorator T820 (Genetronics, Inc., San Diego, Calif.), a PA-2000 (Cyto Pulse Sciences, Inc., Columbia, Md.) or a PA-4000, (Cyto Pulse Sciences, Inc., Columbia, Md.). These signal generators and methods of using them are described in U.S. Pat. Nos. 6,314,316, 6,241,701, 6,233,482, 6,135,990, 5,993,434, and 5,704,908.

The electrodes may be activatable in a predetermined sequence, which may include sequential or simultaneous activation of any or all of the electrodes. Suitable devices can be used, for example, with alternating current, direct current, pulsed alternating current, pulsed direct current, high- and low-voltage alternating current with variable frequency and amplitude, variable direct current waveforms, variable alternating current signals biased with variable direct current waveforms, variable alternating current signals biased with constant direct current, and square wave pulse signals. Selective control of the application of electrical signals between the individual electrodes can be accomplished for example, manually, mechanically, or electrically.

### EXAMPLES

The following examples are offered to illustrate, but not to limit the claimed invention.

### Example 1

### Materials and Methods

Intraductal instillation of a DNA construct to the rat submandibular glands: Male Sprague-Dawley rats (weighing 260-280 g) were fasted overnight prior to experiment. After anesthesia with i.m. injection of mixture of ketamine (30 mg/kg body weight (b.wt.)), xylazine (6.0 mg/kg b.wt.) and acepromazine (1.0 mg/kg b.wt.), both right and left salivary gland ducts were cannulated with a fine polyurethane tubing (i.d.0.005″) and cemented in place with a small drop of Krazyglue. Atropine was then administered subcutaneously (0.5 mg/kg b.wt.) and, after 10 minutes, 200 μl of 0.9% NaCl containing 175 μg of luciferase or SEAP encoding plasmid DNA with or without formulants was injected retrogradely into each gland through a syringe pump with a constant flow rate. The tubing was kept in place for 10 additional minutes after injection.

DNA electrotransfer by electroporation in rat submandibular salivary glands: Immediately after DNA delivery into salivary glands, animals to be treated with electroporation were placed in a supine position. The left and right submandibular, salivary gland were visualized by marking the skin surface with a permanent marker on the anterior side of the neck. The work area was washed down with ethanol. A sterile 'BTX 2-needle Array Electrode was inserted transcautaneously into the center of each gland to a depth of 2-4 mm. Electric pulses generated by a ECM 830 electroporator (BTX Instrument Division, San Diego, Calif.) were applied to glands through this electrode. Electrical contact with gland tissue through the skin was ensured by shaving the work area and applying a conductive gel. Different electrical parameters, e.g., field strength, duration, frequency, and total number of applied pulses, were tested to maximize gene delivery while minimizing irreversible cell damage. After electroporation, all animals were monitored under a heat lamp until awakening.

Collection of salivary gland tissue and blood samples to assay for transgene expression: 48 hours post electroporation mediated gene transfer, the rats were anesthetized by intraperitoneal injection of pentobarbital (50 mg/kg body weight), blood was collected by cardiac puncture and both submandibular glands were removed. Salivary gland tissues were homogenized in 1.0 ml cold Luciferase Lysis Buffer (Becton Dickinson, San Jose, Calif.) per 0.1 g tissue. All homogenized tissue lysates and blood samples were analyzed for reporter gene expression.

Measurement of Luciferase activity: Luciferase activity in homogenized salivary gland tissue was determined using the Enhanced Luciferase Assay Kit (BD PharMingen) and the Mono light 2010 luminometer (Analytical Luminescence Laboratories). Light emissions were measured over a 10 second period and activity is expressed as Relative Light Units, a function of Luciferase activity.

Measurement of secreted alkaline phosphatase (SEAP) enzyme activity: SEAP activity in homogenized salivary gland tissue or plasma was measured using the SEAP Reporter Gene Assay (Roche) that employs the disodium 3-(4-metho xyspiro {1,2-dioxetane3,2-(5-chloro) tricyclo [3.3.1.1^{3,7}] decan}-4-yl)phenyl phosphate (CSPD) chemiluminescent substrate (Tropix). CSPD conversion by SEAP results in formation and decomposition of the dioetance anion emitting chemiluminescent light. Light signals were recorded using the L Max luminometer (Molecular Devices) and expressed as Relative Light Units, a function of SEAP activity.

### Example 2

### Electrical Salivary Gland Stimulation Increases Gene Transfection Efficiency

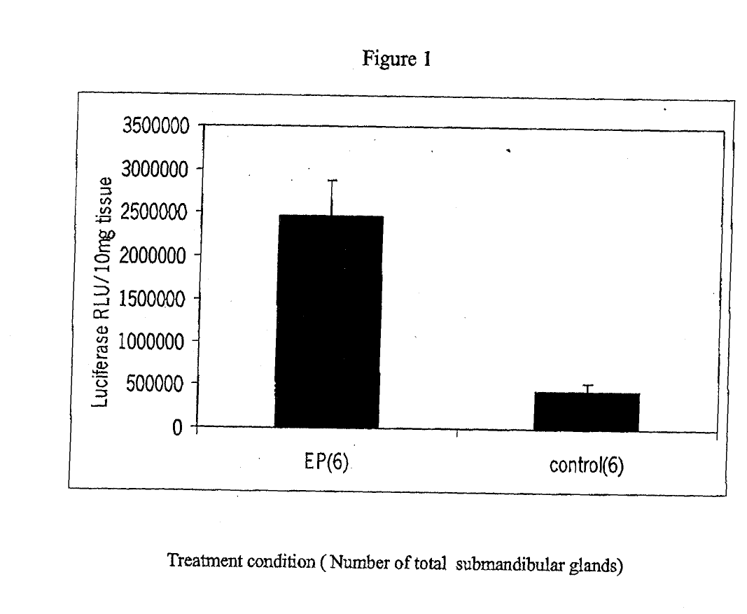

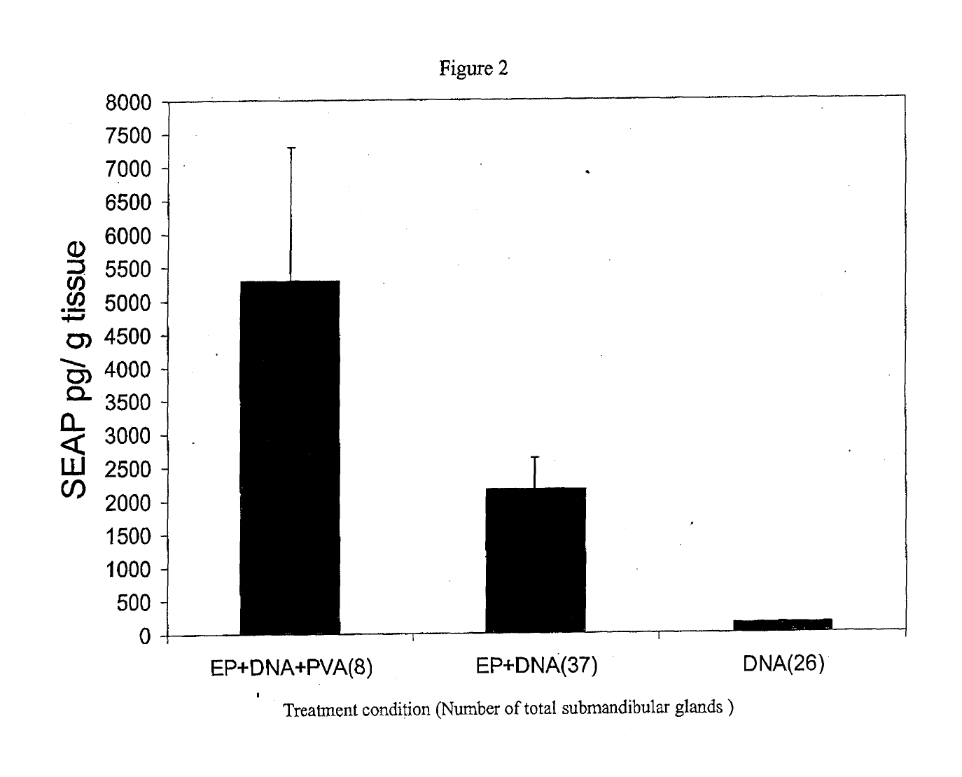

As shown in FIG. 1, expression of luciferase in the rat submandibular gland is enhanced by electroporation as compared to control. On average, the values obtained were approximately 5-6 times greater than the levels of expression in the control group. Additionally, data presented in FIG. 2 demonstrates that electroporation enhances SEAP expression by 50-fold. Together, FIGS. 1 and 2 demonstrate that electroporation can be used to enhance transfection efficacy of different reporter genes in the same target organ.

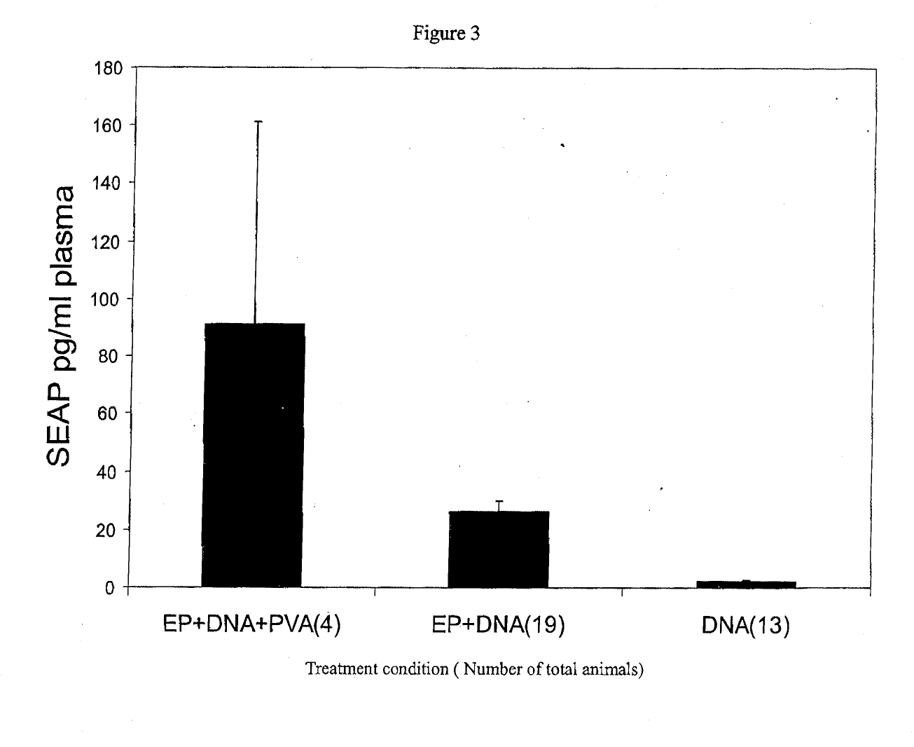

The data presented in FIG. 3 indicates that higher SEAP protein secretion into blood circulation was observed after electroporation-mediated gene transfer in rat submandibular salivary glands. This salivary gland electro-transfer method provides 40 to 50-fold increase in SEAP protein secretion, compared to simple plasmid DNA injection. FIG. 3 also demonstrates that methods of in vivo non-viral gene transfer that do not include a step of electroporating produce very low or no secreted protein into blood circulation (control values).

### Example 3

### Electrode Configuration Influences Transgene Expression in Rat Submandibular Salivary Glands

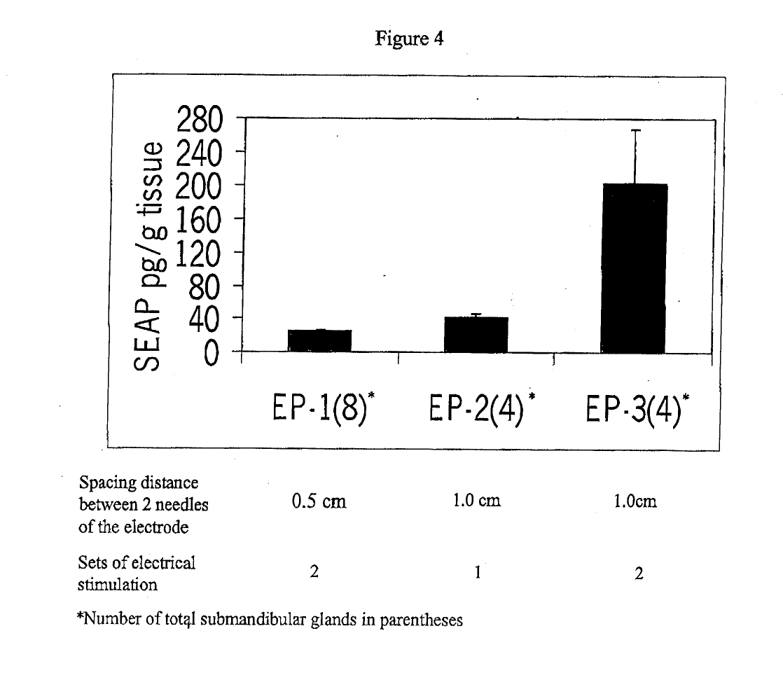

As shown in FIG. 4, for electroporation-mediated gene transfer experiments, different levels of transgene expression were observed using an electrode with varied configurations. The effect of the spacing distance between 2 needles of the same electrode on transgene expression from salivary glands was compared. SEAP activity was approximately 10-fold higher by using an electrode with 1 cm spacing distance between 2 needles than by using an electrode with 0.5 cm spacing distance between 2 needles.

### Example 4

### Effect of Polyvinyl Alcohol on Electroporation Mediated Gene Transfer in Rat Submandibular Salivary Glands

Data presented in FIGS. 2 and 3 demonstrate that polyvinyl alcohol (PVA) can further increase electroporation-mediated gene transfer efficiency in the rat submandibular glands. PVA is a polymer commonly used for controlled drug delivery and has been shown to enhance transfection efficiency in other in vivo models (intramuscular). An increase of transgene expression in both salivary gland tissue and blood circulation was achieved in the presence of PVA in DNA containing solutions. The enhanced effects of transgene expression was higher (five-fold increase) in blood circulation than that in gland tissue(two-fold increase).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for transfecting cells, the method comprising the steps of:
administering a nucleic acid to salivary gland; and
electroporating the salivary gland,
wherein the step of electroporating comprises contacting the salivary gland with an electrode comprising 2 needles and pulsing the salivary gland.

2. The method of claim 1, wherein the step of administering is by cannulation.

3. The method of claim 1, wherein the step of administering is by injection.

4. The method of claim 1, wherein the step of administering is via a salivary gland duct.

5. The method claim 1, wherein the salivary gland is a submandibular salivary gland.

6. The method of claim 1, wherein the salivary gland is a parotid salivary gland.

7. The method of claim 1, wherein the salivary gland is a sublingual salivary gland.

8. The method of claim 1, wherein the nucleic acid is operably linked to an expression control sequence.

9. The method of claim 8, wherein the nucleic acid encodes secreted alkaline phosphatase.

10. The method of claim 8, wherein the nucleic acid encodes luciferase.

11. The method of claim 8, wherein the nucleic acid encodes a therapeutic protein.

12. The method of claim 11, wherein the therapeutic protein is growth hormone.

13. The method of claim 11, wherein the therapeutic protein is insulin.

14. The method of claim 11, wherein the therapeutic protein is clotting factor VIII.

15. The method of claim 11, wherein the therapeutic protein is clotting factor IX.

16. The method of claim 11, wherein the therapeutic protein is erythropoietin.

17. The method of claim 11, wherein the therapeutic protein is calcitonin.

18. The method of claim 11, wherein the therapeutic protein is α-galactosidase.

19. The method of claim 11, wherein the therapeutic protein is α-glucosidase.

20. The method of claim 11, wherein the therapeutic protein is glucocerebrosidase.

21. The method of claim 11, wherein the therapeutic protein is immunoglobulin.

22. The method of claim 1, wherein the needles are about 1 cm apart.

23. The method of claim 1, wherein the needles are about 0.5 cm apart.

24. The method of claim 1, wherein the electrode emits an electric field strength from about 1 to about 1000 V/cm and a pulse length from about 1 to about 60 ms.

25. The method of claim 1, wherein the step of pulsing comprises from about 1 to about 30 pulses.

26. The method of claim 1, wherein the step of pulsing comprises from about five pulses to about six pulses.

27. The method of claim 1, wherein the electrode emits an electric field strength from about 100 V/cm to about 200 V/cm and an electrical pulse length from about 10 ms to about 20 ms.

28. The method of claim 1, wherein the step of electroporating further comprises:
repositioning the electrode in a second position; and
pulsing the salivary gland.

29. The method of claim 28, wherein the electrode emits an electric field strength from about 1 to about 1000 V/cm and a pulse length from about 1 to about 60 ms.

30. The method of claim 28, wherein the step of pulsing comprises from about 1 to about 30 pulses.

31. The method of claim 28, wherein the step of pulsing comprises from about five pulses to about six pulses.

32. The method of claim 28, wherein the electrode emits an electric field strength from about 100 V/cm to about 200 V/cm and an electrical pulse length from about 10 ms to about 20 ms.

33. The method of claim 1, wherein the step of electroporating further comprises:
contacting the salivary gland with a second electrode; and
pulsing the salivary gland.

34. The method of claim 33, wherein the first electrode is in a first position and the second electrode is in a second position.

35. The method of claim 33, wherein the steps of contacting are sequential.

36. The method of claim 33, wherein the steps of contacting are simultaneous.

37. The method of claim 28, wherein the first and second electrodes emit an electric field strength from about 1 to about 1000 V/cm and a pulse length from about 1 to about 60 ms.

38. The method of claim 28, wherein the step of pulsing comprises from about 1 to about 30 pulses.

39. The method of claim 28, wherein the step of pulsing comprises from about five pulses to about six pulses.

40. The method of claim 28, wherein the first and second electrodes emit an electric field strength from about 100 V/cm to about 200 V/cm and an electrical pulse length from about 10 ms to about 20 ms.

41. The method of claim 1, wherein a formulant is administered with the nucleic acid.

42. The method of claim 41, wherein the formulant is a member selected from the group consisting of: divalent transition metal compounds, polyanionic compounds, and peptides.

43. The method of claim 41, wherein the formulant is a divalent transition metal compound.

44. The method of claim 43, wherein the divalent transition metal compound is a member selected from the group consisting of zinc halide, zinc oxide, zinc selenide, zinc telluride, zinc sulfate, zinc acetate, and zinc chloride

45. The method of claim 42, wherein the divalent transition metal compound is zinc chloride.

46. The method of claim 42, wherein the polyanionic compound is poly-L-glutamate.

47. The method of claim 41, wherein the formulant is polyvinyl alcohol.
